(19) United States
(12) Patent Application Publication
(10) Pub. No.: US 2022/0328156 A1
(43) Pub. Date: Oct. 13, 2022

(54) METHOD, APPARATUS, DEVICE AND MEDIUM FOR MEDICATION DECISION SUPPORT BASED ON GRAPHICS STATE MACHINE

(71) Applicant: ZHEJIANG UNIVERSITY, Hangzhou (CN)

(72) Inventors: Dongsheng HONG, Hangzhou (CN); Xiaojian LIU, Hangzhou (CN); Xiaoyang LU, Hangzhou (CN); Zhe GAO, Hangzhou (CN); Tingting CHEN, Hangzhou (CN); Jian NI, Hangzhou (CN)

(21) Appl. No.: 17/841,688

(22) Filed: Jun. 16, 2022

Related U.S. Application Data
(63) Continuation of application No. PCT/CN2022/070480, filed on Jan. 6, 2022.

(30) Foreign Application Priority Data
Apr. 10, 2021 (CN) 202110385599.5

(51) Int. Cl. G16H 20/10; G16H 10/60; G16H 70/40; G16H 50/70; G06F 40/30; G06F 40/279; G06F 40/242

(52) U.S. Cl. CPC G16H 20/10; G16H 10/60; G16H 70/40; G16H 50/70; G06F 40/30; G06F 40/279; G06F 40/242

(57) ABSTRACT

Provided is a medication decision support method and apparatus based on a graphics state machine. The method comprises the steps of: acquiring a medication consultation statement of a user; extracting a symptom information entity, an allergy information entity and an disease onset information entity in the medication consultation statement; forming a graphics state machine set with a high response speed to a target event; analyzing the disease degree information in the medication consultation statement of the user and an emotion word segmentation dictionary, and comparing the disease degree information with the disease degree information spoken to obtain a corrected value of the disease degree; removing excessive judgment or underestimated judgment, on the disease degree, and obtaining accurate disease degree information; and achieving medication decision support aiming at accurate disease degree information through the medication decision support device.

### CROSS-REFERENCE TO RELATED APPLICATIONS

The present application is a continuation of International Application No. PCT/CN2022/070480, filed on Jan. 6, 2022, which claims priority to Chinese Application No. 202110385599.5, filed on Apr. 10, 2021, the contents of both of which are incorporated herein by reference in their entireties.

### TECHNICAL FIELD

The present disclosure relates to the field of rational medication, in particular to a medication decision support method and device based on a graphics state machine.

### BACKGROUND

Drugs refer to substances that are used for the prevention, treatment and diagnosis of human diseases, for the purpose of regulating human physiological functions and for which indications or functional indications, usage and dosage are specified, including Chinese medicinal materials, Chinese herbal pieces, Chinese patent medicine, chemical raw materials and their preparations, antibiotics, biochemical drugs, radioactive drugs, serum, vaccines, blood products and diagnostic drugs. It is well known that the application of drugs has played a positive role in improving people's health. However, it must be recognized that drugs have two sides, and the method of use, quantity, time and other factors determine the therapeutic effect to a large extent. Misuse can not only “cure diseases”, but may “cause diseases”, or even endanger the life safety of patients.

With the gradual improvement of drug accessibility, quality and efficacy in China, drugs have become more and more accessible, so whether their use is safe and reasonable has become a key factor affecting public drug safety. In view of how to use drugs safely and reasonably, a variety of auxiliary decision-making systems for clinical medication have been developed in the prior art. The Chinese patent ZL201110452960.8 discloses a decision support method for clinical rational medication, which includes a database of clinical rational medication specifications and a variety of discrimination units, which are embedded in a HIS (Hospital Management Information System) in the form of controls. It realizes the functions of automatically giving treatment plans, automatically monitoring drugs that may be allergic, automatically reviewing drug compatibility taboos and interactions, etc., and plays a role in helping doctors optimize medication plans. However, this decision support method is based on “Clinical Rational Medication Specification Database”, which is a static database structure, and there may be a problem of slow response (stagnation) in the actual use process; in addition, the patented method includes a variety of discrimination units, which are complex in composition, and a lot of information often pops up in the doctor advice interface. Too much information will easily interfere with the doctor's judgment, and it is not suitable for the public to discriminate. On the other hand, the existing clinical rational medication decision support methods adopt the evidence of a group level through a static database, and provide the evidence level based on probability, which easily leads to the situation that multiple homogeneous patients are recommended with one medication plan, which lacks the intelligent evaluation for the individual levels of patients.

Therefore, with the development of artificial intelligence technology, it is necessary to improve the existing technology, realize the intelligent judgment of patients' individual level in the field of rational medication, break through the limitation of group evidence, and provide information support for medication reference solutions for the clinic that fully consider individuality of the patients.

### SUMMARY

In order to solve the technical problem of rational medication, the present disclosure provides a medication decision support method and apparatus based on a graphics state machine, which can realize the intelligent judgment of the disease severity of a patient, and enable a rational medication system to only give the most critical information about medication by correlating the specific dosage and frequency of drug administration: how to take the drugs, thus avoiding the user's choice obstacle caused by complicated information.

By fusing unstructured text information and coded data into a structured descriptive graph-spectrum model, the graphics state machine can update the rules of rational medication in the form of graph editing, thus getting rid of the trouble that medical staff are not good at code editing, and effectively improving the update efficiency of rational medication knowledge.

From the point of view of the present disclosure, firstly, a graphics state machine model is adopted to form a graphics state machine set indicating medication decision suitable for drug recommendation, and the target and efficiency of drug recommendation are improved through the linkage between graph machines; secondly, emotional discrimination and neural network models are introduced to score emotions according to condition descriptions of different patients, so as to correct patients' emotional expression errors.

Therefore, the key of the present disclosure lies in the following:

(1) The graphics state machine of the present disclosure is different from the prior art. The graphics state machine set of the present disclosure realizes the association of different age levels, weight, height, onset time points, onset symptoms with medication recommendations. The medication recommendation includes specific medication frequency, medication dosage and medication times, so as to obtain accurate decisions and improve the accuracy of medication recommendations. The design of the three graphics state machines in series improves the target of medication recommendation and operation efficiency of the system.

(2) The present disclosure proposes an emotional score discrimination method based on the consultation statement of the patient, which realizes the intelligent judgment of the patient's disease severity at individual level through the correlation between the emotional score and patient's disease severity.

(3) The present disclosure uses the customized neural network model to analyze the disease degree of the statement input by the user, and then compares it with the disease degree described by the user to obtain the corrected value of the disease degree, and uses the corrected value to remove the excessive judgment or underestimation of the disease degree caused by the emotional factors of the user, so as to realize the accurate judgment for the condition description of the patient.

According to an aspect of the present disclosure, a medication decision support method based on a graphics state machine is provided, which includes:

acquiring a medication consultation statement of a user;

extracting a symptom information entity, an allergy information entity and an disease onset information entity from the medication consultation statement through word segmentation and semantic recognition

generating medication decision information by using the symptom information entity, the allergy information entity and the disease onset information entity based on a preset medication decision indicating graphics state machine set. The the medication decision indicating graphics state machine set includes at least one medication decision indicating graphics state machine;

providing primary available drugs for the user according to the medication decision information.

The allergy information entity includes an allergic drug entity and an allergic food entity.

The disease onset information entity includes an age level entity, a weight entity, a height entity, an onset time entity and a disease severity entity.

The medication decision information includes drug name information, drug dose information and medication frequency information.

In a preferred embodiment, the medication decision indicating graphics state machine set includes a first medication decision indicating graphics state machine, a second medication decision indicating graphics state machine and a third medication decision indicating graphics state machine.

The first medication decision indicating graphics state machine includes the symptom information entity, a diagnosis entity, a drug indication entity, and a symptom-diagnosis-drug indication correlation.

The second medication decision indicating graphics state machine includes the allergic drug entity, the allergic food entity, a correlation of cross allergy between drugs, and a correlation of cross allergy between drugs and food.

The third medication decision indicating graphics state machine includes the age level entity, the weight entity, the height entity, the onset time entity, the disease severity entity, and a correlation between an age level, a weight, a height, an onset time, a disease degree and a dosage and a medication frequency.

The first, second and third medication decision indicating graphics state machines have a plurality of common entities. The medication decision graphics state machine includes a definition of different clinical event entities, a definition of multiple attributes of the entities and a correlation between the entities; the graphics state machine is preset with general medical logic processors, and each general medical logic processor contains an applicable logic; the relationship between the entities in the medication decision graphics state machine can be inferred based on the applicable logic, and a medication recommendation result can be obtained according to individual inputs defined by the multiple attributes of the entities.

Specifically, the disease onset information entity includes disease severity information, and the method further includes:

Analyzing medication consultation statement of the user based on the emotion word segmentation dictionary, and generating an emotional score of the consultation statement. The emotional score is divided into different emotional tendency grades, which is specifically as follows:

The medication consultation statement of the user is divided into an emotional verb W_{V} and an emotional adverb W_{adj} based on the emotion word segmentation dictionary; the current emotional verb W_{V} is matched with the emotion dictionary, if it is a positive word, an emotional value is 1, if it is a negative word, the emotional value is −1; the emotional adverb W_{adj} is also matched with the emotion dictionary, and if it is a positive word, the emotional value is 1, if it is a negative word, the emotional value is −1.

A cumulative tendency score of each emotional verb W_{V} is calculated:

$$α = {\sum }_{i = 1}^{n} W v i \qquad ( 1 )$$

where α>0 indicates that an action emotional tendency is of a positive feedback type (positive-α), α<0 indicates that the action emotional tendency is of a negative feedback type (negative-α); α=0 indicates that the action emotional tendency is of a neutral type (neutral-α).

A cumulative tendency score of each emotional adverb W_{adj} is calculated:

$$β = {\sum }_{i = 1}^{n} Wadji \qquad ( 2 )$$

where β>0 indicates that an action emotional tendency is of a positive feedback type (positive-β); β<0 indicates that the action emotional tendency is of a negative feedback type (negative-β); β=0 indicates that the action emotional tendency is of a neutral type (neutral-β).

Generating a specific emotional tendency grade and a corresponding emotional score according to the expressions of the emotional verb W_{V} and the emotional adverb W_{adj}; which is specifically as follows:

| | Emotional | Emotional | |
|---|---|---|---|
| Applicable conditions | grade | score | Remarks |
| α > and β > 0 | 1 | 0.5 | See steps (1) |
| α > 0 and β < 0 and | 2 | 0.3 | and (2) above |
| \|α\| > \|β\| | | | for the specific |
| α > 0 and β < 0 and | 3 | 0.1 | recognition |
| \|α\| < \|β\| | | | algorithm. |
| either of the conditions | 4 | 0 | |
| (1) or (2) is satisfied: | | | |
| (1)α > 0 and β < 0 and | | | |
| \|α\| = \|β\| | | | |
| (2)α > 0 and β < 0 and | | | |
| \|α\| = \|β\| | | | |
| α < 0 and β > 0 and | 5 | −0.1 | |
| \|α\| < \|β\| | | | |
| α < and β > 0 and | 6 | −0.3 | |
| \|α\| > \|β\| | | | |
| α < 0 and β < 0 | 7 | −0.5 | |

Correcting the disease severity information by using the emotional score.

In a preferred embodiment, the step of correcting the disease severity information by using the emotional score includes:

Generating estimated disease severity information according to the emotional score, which includes the following steps of: calculating the estimated disease severity information by using historical diagnosis data of hospital electronic cases in recent N years, wherein the estimated disease severity information includes mild diseases, moderate diseases, severe diseases and critical diseases, and each estimated disease severity information is obtained by a decision tree generated by the electronic case data of related diagnosis, and further decision tree nodes are distinguished by emotional tendency grades.

Comparing the estimated disease severity information with the disease severity information in the medication consultation statement of the user to generate an emotion corrected value; and

Correcting the disease severity information according to the emotion corrected value.

In a preferred embodiment, the step of comparing the estimated disease severity information with the disease severity information in the medication consultation statement of the user to generate an emotion corrected value includes:

Comparing the estimated disease severity information with the disease severity information in the medication consultation statement of the user to generate a difference score.

Generating the emotion corrected value based on the difference score and a disease degree of the disease severity information in the medication consultation statement of the user.

In a preferred embodiment, the step of correcting the disease severity information according to the emotion corrected value includes:

Inputting the emotion corrected value and the disease severity information in the medication consultation statement of the user into a neural network model corresponding to the target user, wherein an output of the neural network model is the estimated disease severity information.

The neural network model is trained by using historical consultation statements of patients with the same diagnosis as the user and actual disease severity information; the disease severity information includes mild, moderate, severe and critical degrees; the patients with the same diagnosis include patients with a same diagnosis, a same age level and a same education level.

Preferably, the step of training the neural network model by using historical consultation statements of patients with the same diagnosis as the user and actual disease severity information includes:

De-noising the historical consultation statements of the patients with the same diagnosis, the age level and education level as the target user.

Performing emotion analysis on the denoised historical consultation statements based on the emotion dictionary to obtain corresponding emotional scores; wherein the emotion dictionary is obtained by expanding a Hownet emotion dictionary and simplified Chinese NTUSD dictionary by combining a basic emotion dictionary, and a method of expanding the emotion dictionary is mainly based on semantic similarity and synonym methods.

Marking the corresponding historical consultation statements with the emotional scores, and forming training data by combining the actual disease severity information.

Training the neural network model by using a training set including a plurality of the training data.

In a second aspect of the present disclosure, a medication decision support apparatus based on a graphics state machine is provided, including:

A user medication consultation statement acquisition processor configured to acquire a user medication consultation statement.

A semantic analyzer configured to extract a symptom information entity, an allergy information entity and an disease onset information entity from the medication consultation statement through word segmentation and semantic recognition.

A medication decision information generator configured to generate medication decision information by using the symptom information entity, the allergy information entity and the disease onset information entity based on a preset medication decision indicating graphics state machine set, wherein the medication decision indicating graphics state machine set includes at least one medication decision indicating graphics state machine.

A medication support processor configured to provide primary available drugs for the user according to the medication decision information.

The allergy information entity includes an allergic drug entity and an allergic food entity.

The disease onset information entity includes an age level entity, a weight entity, a height entity, an onset time entity and a disease severity entity.

The medication decision information includes drug name information, drug dose information and medication frequency information.

In a third aspect of the present disclosure, an operation method of a medication decision support apparatus based on a graphics state machine is provided, including: obtaining a medication consultation statement of a user through voice input or graphic input, and then converting the medication consultation statement into entity participles by a semantic analyzer, and correcting by an emotional score and neural network model; taking this as input, using the first medication decision indicating graphics state machine to determine the drug names of various available drugs, which are input to the second medication decision indicating graphics state machine, optimizing the drugs to be administered according to the patient's allergy entity information; and then further inputting to the third medication decision indicating graphics state machine to obtain medication decision information; wherein the decision information includes drug name, drug dose and medication frequency.

In a fourth aspect of the present disclosure, provided is a medication decision support device based on a graphics state machine, comprising a memory, a processor, a communication processor, and a computer program stored in the memory and operable on the processor, wherein the processor implements the medication decision support method based on a graphics state machine when executing the program.

In a fourth aspect of the present disclosure, provided is a computer-readable storage medium on which a computer program is stored, wherein the computer program, when executed by a processor, realizes the medication decision support method based on a graphics state machine.

The beneficial effects of the present disclosure include: providing a medication decision support method, apparatus, device and medium based on a graphics state machine. Through the association of three independent first, second and third medication decision indicating graphics state machines, a set of graphics state machines with a fast response speed to target events is formed. The graphics state machines complete the update of knowledge in the field of rational medication in the form of graphic editing without code updating, which also enables the medical staff to edit reasoning rules of rational medication by themselves without programmers. In addition, the constructed graphics state machine set can fully consider the influence of disease information on drug dose and medication frequency through emotional scores, so as to improve the accuracy of drug recommendation. Further, the method and device of the present disclosure can analyze the disease degree of the statement input by the user by using the emotion discrimination and customized neural network model, and then compare it with the disease degree spoken by the user to obtain the corrected value of the disease degree, and use the corrected value to remove the excessive judgment or underestimation of the disease degree caused by the emotional factors of the user, so as to realize the intelligent judgment of the disease degree irrelevant to the individual cognitive level of the patient.

### BRIEF DESCRIPTION OF DRAWINGS

In order to more clearly explain the embodiments of the present disclosure or the technical solutions in the prior art, the drawings that need to be used in the description of the embodiments or the prior art will be briefly introduced below. Obviously, the drawings in the following description are only some embodiments of the present disclosure. For those of ordinary skill in the art, other drawings can be obtained according to these drawings without any creative effort.

FIG. 1 is a structural schematic diagram of a medication decision indicating graphics state machine set according to an embodiment of the present disclosure;

FIG. 2 is a man-machine doctor advice review mode based on the graphics state machine medication decision support method according to an embodiment of the present disclosure;

FIG. 3 is a schematic diagram of the maintenance process of medication warning information rules in the medication decision support method based on a graphics state machine according to an embodiment of the present disclosure;

FIG. 4 is a structural schematic diagram of a medication decision support apparatus based on a graphics state machine according to an embodiment of the present disclosure;

FIG. 5 is a structural schematic diagram of an electronic device of a medication decision support apparatus based on a graphics state machine according to an embodiment of the present disclosure.

### DESCRIPTION OF EMBODIMENTS

The technical solutions in the embodiments of the present disclosure will be clearly and completely described below with reference to the drawings in the embodiments of the present disclosure. Obviously, the described embodiments are only part, not all of the embodiments of the present disclosure. Based on the embodiments of the present disclosure, all other embodiments obtained by those skilled in the art without creative effort belong to the scope of protection of the present disclosure.

At present, with the basic solution of drug accessibility and the gradual improvement of drug quality and curative effect in China, whether the medication sector is safe and reasonable has become an important factor affecting the drug safety for the public. Therefore, how to make good use of drugs is the key to solve the problem of public drug safety.

Based on this, the first aspect of the present disclosure is to provide a medication decision support method based on a graphics state machine, which includes:

S01: acquiring a medication consultation statement of a user.

S02: extracting a symptom information entity, an allergy information entity and an disease onset information entity from the medication consultation statement through word segmentation and semantic recognition.

S03: generating medication decision information by using the symptom information entity, the allergy information entity and the disease onset information entity based on a preset medication decision indicating graphics state machine set, wherein the medication decision indicating graphics state machine set includes at least one medication decision indicating graphics state machine.

Specifically, the graphics state machine for medication decision integrates unstructured text information and coded data into a structured entity attribute description model, and the medication rules can be updated through graphic editing, thus avoiding complicated code operations. The introduction of graphics state machine technology in the field of rational medication can effectively improve the speed and response efficiency of knowledge updating in the field of rational medication. In the graphics state machine of clinical medical knowledge representation, it includes the definition of different clinical event entities, the definition of multiple attributes of entities and the relationship between the entities. In addition, the graphics state machine can be preset with general medical logic processors. Specifically, the graphics state machine for medication decision in this application can be preset with general medical logic processors based on clinical guidelines and drug instructions. Each general medical logic processor contains applicable medical logic, and the relationship between entities in the graphics state machine can be inferred based on the applicable medical logic. Furthermore, a drug recommendation result can be obtained according to the individual inputs defined by multiple attributes of the entities.

S04: providing primary available drugs for the user according to the medication decision information.

The allergy information entity includes an allergic drug entity and an allergic food entity.

The disease onset information entity includes an age level entity, a weight entity, a height entity, an onset time entity and a disease severity entity.

The medication decision information includes drug name information, drug dose information and medication frequency information.

Based on the preset medication decision graphics state machine set, the present disclosure uses the acquired symptom information entity, allergy information entity and disease onset information entity to generate medication decision information, and fully considers the influence of disease information on drug dose and medication frequency, thereby improving the accuracy of medication recommendation, better supporting users for medication, and simultaneously improving the therapeutic effect of drugs.

Concretely, after obtaining the medication consultation statement, the medication consultation statement is segmented, and all possible words matched with the lexicon are segmented. Then, a statistical language model is used to determine the optimal segmentation result, and then part-of-speech tagging is performed to generate a symptom information entity, an allergy information entity and a disease onset information entity, and medication decision information is generated according to this information. The symptom information entity include fever, pain, dizziness, dyspnea, etc. The allergy information entity includes allergic drugs, food or allergic situations (such as mental, emotional excitement or exposure to sunlight), etc. The disease onset information entity includes age, height, weight, onset time, past medical history and family medical history, etc. The medication decision indicating graphics state machine includes unstructured, semi-structured and structured drug knowledge and related diagnosis and medication data of various diseases, and drug knowledge includes drug names, ingredients, characteristics, indications, functional indications, specifications, dosage, adverse reactions, taboos, precautions, drug interactions, drug toxicology and other information.

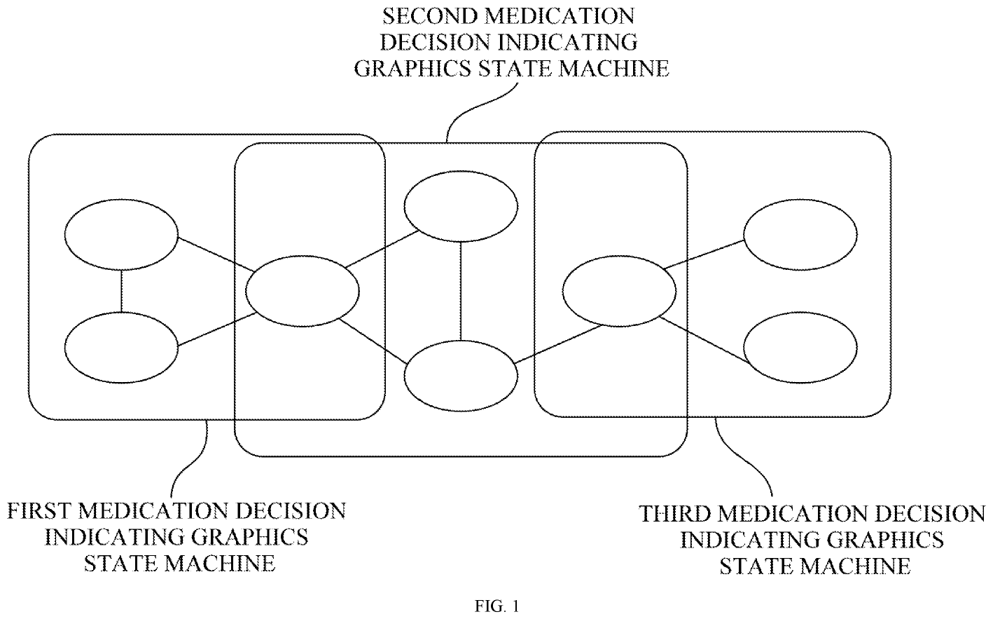

In some preferred embodiments, as shown in FIG. 1, the medication decision indicating graphics state machine set includes a first medication decision indicating graphics state machine, a second medication decision indicating graphics state machine and a third medication decision indicating graphics state machine.

The first medication decision indicating graphics state machine includes the symptom information entity, a diagnosis entity, a drug indication entity, and a symptom-diagnosis-drug indication correlation.

The second medication decision indicating graphics state machine includes the allergic drug entity, the allergic food entity, a correlation of cross allergy between drugs, and a correlation of cross allergy between drugs and food.

The third medication decision indicating graphics state machine includes the age level entity, the weight entity, the height entity, the onset time entity, the disease severity entity, and a correlation between an age level, a weight, a height, an onset time, a disease degree and a dosage and a medication frequency.

The first, second and third medication decision indicating graphics state machines have a plurality of common entities.

Specifically, as for the construction of the medication decision indicating graphics state machine, by starting from the most primitive data (including structured, semi-structured and unstructured data), the knowledge elements related to medication decision, such as the knowledge of various drugs, the relationship between drugs, the information of various diseases, the relationship between various diseases and drugs and the like are extracted through knowledge extraction technology, and then the knowledge elements related to medication decision are further processed through certain effective means, and further expanded through knowledge fusion and knowledge reasoning to form a high-quality medication decision indicating graphics state machine.

In addition, through the setting of multiple common entities, graphs and entities can be quickly found from the common attributes of multiple state machines, avoiding the graph retrieval of a single state machine one by one, thus improving the retrieval efficiency and retrieval target.

For example, when antibiotics are recommended to a person that is positive in a penicillin skin test, first a category attribute, antibiotics, is found through the graph. Then cephalosporin instead of penicillin is found from the antibiotic matching weight, because there is the attribute in the penicillin graph that penicillin cannot be used for a person positive in penicillin skin test.

Then the list of recommended drugs can be found through the workflow of the state machine.

Specifically, the disease onset information entity includes disease severity information, and the method further includes:

Analyzing medication consultation statement of the user based on the emotion word segmentation dictionary, and generating an emotional score of the consultation statement, wherein the emotional score is divided into different emotional tendency grades, which is specifically as follows:

The medication consultation statement of the user is divided into an emotional verb W_{V} and an emotional adverb W_{adj} based on the emotion word segmentation dictionary; the current emotional verb W_{V} is matched with the emotion dictionary, if it is a positive word, an emotional value is 1, if it is a negative word, the emotional value is −1; the emotional adverb W_{adj} is also matched with the emotion dictionary, and if it is a positive word, the emotional value is 1, if it is a negative word, the emotional value is −1.

A cumulative tendency score of each emotional verb W_{V} is calculated:

α=Σᵢ₌₁ⁿWvi (1)

where α>0 indicates that an action emotional tendency is of a positive feedback type (positive-α), α<0 indicates that the action emotional tendency is of a negative feedback type (negative-α); α=0 indicates that the action emotional tendency is of a neutral type (neutral-α).

A cumulative tendency score of each emotional adverb W_{adj} is calculated:

β=Σᵢ₌₁ⁿWadji (2)

where β>0 indicates that an action emotional tendency is of a positive feedback type (positive-β); β<0 indicates that the action emotional tendency is of a negative feedback type (negative-β); β=0 indicates that the action emotional tendency is of a neutral type (neutral-β).

Generating a specific emotional tendency grade and a corresponding emotional score according to the expressions of the emotional verb W_{V} and the emotional adverb W_{adj}; which is specifically as follows:

| | Emotional | Emotional | |
|---|---|---|---|
| Applicable conditions | grade | score | Remarks |
| α > and β > 0 | 1 | 0.5 | See steps (1) |
| α > 0 and β < 0 and | 2 | 0.3 | and (2) above |
| \|α\| > \|β\| | | | for the specific |
| α > 0 and β < 0 and | 3 | 0.1 | recognition |
| \|α\| < \|β\| | | | algorithm. |
| either of the conditions | 4 | 0 | |
| (1) or (2) is satisfied: | | | |
| (1)α > 0 and β < 0 and | | | |
| \|α\| = \|β\| | | | |
| (2)α > 0 and β < 0 and | | | |
| \|α\| = \|β\| | | | |
| α < 0 and β > 0 and | 5 | −0.1 | |
| \|α\| < \|β\| | | | |
| α < and β > 0 and | 6 | −0.3 | |
| \|α\| > \|β\| | | | |
| α < 0 and β < 0 | 7 | −0.5 | |

Correcting the disease severity information by using the emotional score.

Specifically, the step of correcting the disease severity information by using the emotional score includes:

Generating estimated disease severity information according to the emotional score.

Comparing the estimated disease severity information with the disease severity information in the medication consultation statement of the user to generate an emotion corrected value; and

Correcting the disease severity information according to the emotion corrected value.

Specifically, the step of generating estimated disease severity information according to the emotional score includes: calculating the estimated disease severity information by using historical diagnosis data of hospital electronic cases in recent N years (e.g., three yeas), wherein the estimated disease severity information includes mild diseases, moderate diseases, severe diseases and critical diseases, and each estimated disease severity information is obtained by a decision tree generated by the electronic case data of related diagnosis, and further decision tree nodes are distinguished by emotional tendency grades.

Specifically, the step of comparing the estimated disease severity information with the disease severity information in the medication consultation statement of the user to generate an emotion corrected value includes:

Comparing the estimated disease severity information with the disease severity information in the medication consultation statement of the user to generate a difference score.

Generating the emotion corrected value based on the difference score and a disease degree of the disease severity information in the medication consultation statement of the user.

Specifically, the disease severity information in the medication consultation statement of the user includes information such as body temperature, heart rate, pulse, blood pressure and disease description emotion. By comparing the information with the normal values of diseases in the medication consultation statement of the user, the disease severity in the medication consultation statement of the user can be deduced, the disease severity in the medication consultation statement of the user is compared with the estimated disease severity information to generate a difference score. The smaller the difference score, it means that the smaller the difference between the disease degree in the medication consultation statement of the user and the estimated disease degree; on the contrary, the larger the difference score, the greater the difference between the disease degree in the medication consultation statement of the user and the estimated disease degree, which requires further information and comparison again.

Specifically, the step of correcting the disease severity information according to the emotion corrected value includes:

inputting the emotion corrected value and the disease severity information in the medication consultation statement of the user into a neural network model corresponding to the target user, wherein an output of the neural network model is the estimated disease severity information.

The neural network model is trained by using historical consultation statements of patients with the same diagnosis as the user and actual disease severity information; the disease severity information includes mild, moderate, severe and critical degrees; the patients with the same diagnosis include patients with a same diagnosis, a same age level and a same education level.

Specifically, the neural network model is a complex network system formed by a large number of simple neurons which are widely connected with each other. The neurons receive the input signals of the emotional score, and these input signals are transmitted through the connection of weights. The total input value received by the neurons will be compared with the threshold value of the neurons, and the neuron output will be generated through the “activation function” processing, and the estimated disease severity information will be output.

Specifically, the step of training the neural network model by using historical consultation statements of patients with the same diagnosis as the user and actual disease severity information includes:

De-noising the historical consultation statements of the patients with the same diagnosis, the age level and education level as the target user.

Performing emotion analysis on the denoised historical consultation statements based on the emotion dictionary to obtain corresponding emotional scores.

Marking the corresponding historical consultation statements with the emotional scores, and forming training data by combining the actual disease severity information.

Training the neural network model by using a training set including a plurality of the training data.

Specifically, the emotion dictionary is obtained by expanding a HOWNET emotion dictionary and simplified Chinese NTUSD dictionary by combining a basic emotion dictionary, and a method of expanding the emotion dictionary is mainly based on semantic similarity and synonym methods;

Specifically, the word segmentation unrelated to emotional words in the historical consultation statements of patients with the same diagnosis, age level and education level of the target users belong to noise data. The historical consultation statements are input into the denoising automatic encoder, the error between the obtained output and the original input signal is calculated, a random gradient descent algorithm is adopted to adjust the weight to minimize the error, the denoised historical consultation statements are output, and then the emotion analysis is carried out to obtain the estimated disease severity information, forming a group of training data, and different consultation statements are input for many times to form multiple sets of different training data.

Specifically, the first medication decision indicating graphics state machine includes a medication warning information entity, a doctor advice information entity, an inspection information entity, an examination information entity, a pathological information entity, an image information entity, and a correlation of drug-medication warning information and a correlation of doctor advice-inspection-examination-pathology-image-drugs.

The second medication decision indicating graphics state machine may also include a doctor's professional level entity, and a correlation between medication warning information and doctor's professional level.

The doctors' professional level entity includes academic qualification, graduation major, practicing department, years of practice and professional title. The attribute information of the doctors' professional level entity can be obtained through hospital personnel files or surveys of doctors, and medication warning information entity can be obtained through drug instructions and medication guides.

Specifically, the correlation between medication warning information and doctors' professional level can be obtained through the following steps: using the medication data of medical electronic cases in hospitals in recent N years (for example, in recent 3 years), using one or more of Beers standard, STOPP/START standard, EU(7)-PIM list and inappropriate medication list published in Chinese literature as a reference, generating the mark of the number of times of irrational medication by the doctors; by taking the number of times of irrational medication as an outcome index, generating an attribute group corresponding to the doctors' professional level by a decision tree or other mathematical models with supervised learning, including various classification combinations of “educational background-graduation major-practice department-practice years-professional title”. Different classification combinations correspond to different entities, and different classification combinations correspond to different doctors' professional levels through the number of times of irrational medication, thus forming the correlation between drug warning information and doctors' professional level.

In addition, the correlation between medication warning information and doctors' professional level can also be obtained by combining academic qualifications, graduation majors, practicing departments, years of practice and doctors' professional titles by an unsupervised learning algorithm or a self-defined method. For the definition, two or more attributes can be selected for definition according to the business needs of medical institutions. For example, according to whether the years of practice exceed 5 years, it is divided into high seniority and low seniority; based on whether a doctor's professional title is intermediate or above, it can be divided into high professional title and low professional title, thus forming the combination of “high seniority-high professional title”, “low seniority-low professional title”, “high seniority-high professional title” and “low seniority-high professional title”. Different combinations can be set for different doctors' professional levels, including junior, intermediate and advanced levels, among which the combination of senior seniority-low professional title and junior seniority-high professional title can all correspond to the intermediate professional level, the combination of junior seniority-low professional title corresponds to junior professional level and the combination of “high seniority and high professional title” corresponds to senior professional level. Among them, the professional level of doctors corresponding to the specific combination classification can also be determined by the form of hospital examination, so as to establish the correlation between medication warning information and doctors' professional level.

The unsupervised learning algorithm can adopt a clustering algorithm.

Specifically, the second medication decision indicating graphics state machine may further include a medication warning information entity, a conflict packet entity for dynamic update of the doctor's operation record, and a correlation between medication warning information and conflict packet information. Wherein, the conflict package entity includes: a past conflict relationship between drugs and diseases where the target doctor makes mistakes, a past conflict relationship between drugs and diagnosis, and a past conflict relationship between drugs and inspections. The conflict entity can be obtained from the comment information about the past doctor's prescription, and the medication operation records that doctors are prone to make mistakes are updated regularly and dynamically, thus forming the correlation between medication warning information and conflict package information.

In addition, the conflict package entities can also be cross-entity networks, specifically: taking information such as inspection, examination, diagnosis, physical signs, drugs, basic information (age, gender, native place) as independent entities, and establishing cross-entity networks, such as cross-entity networks of diagnosis and drugs, inspection and drugs, and examination and drugs.

In the specific implementation process, it can be decided to push the medication warning information of the corresponding warning level to the target doctor by combining the doctor's professional level entity and the conflict package entity.

In some other embodiments, the first medication decision indicating graphics state machine may further include an inspection information entity, an examination information entity, a pathological information entity, an image information entity, and a correlation of inspection-examination-pathology-image-drug.

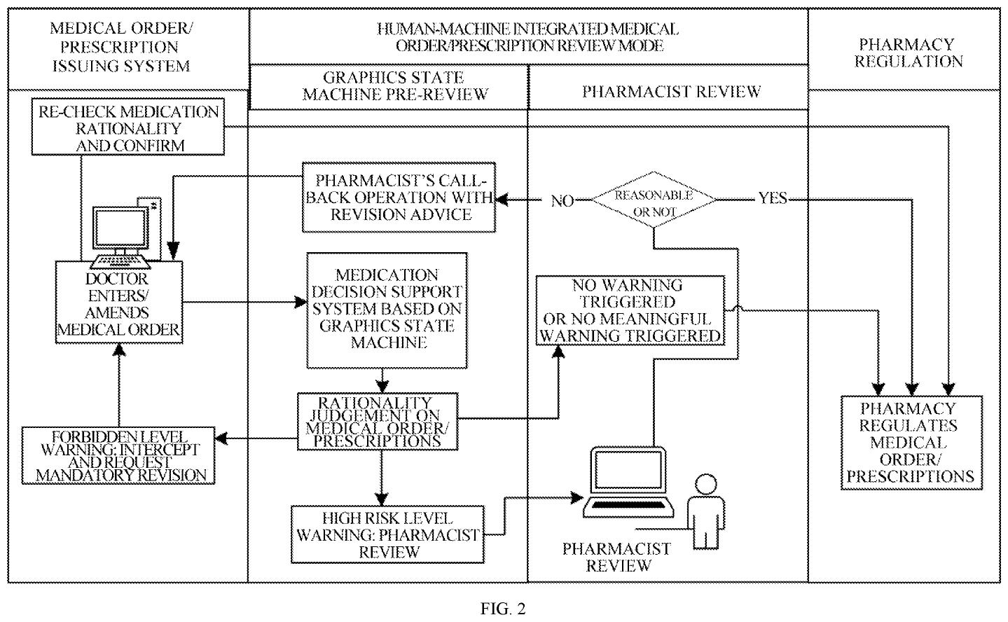

As shown in FIG. 2, this application also provides a man-machine integrated doctor advice review mode based on the medication decision support method based on a graphics state machine. The medication decision support system interprets the medication rules of the graphics state machine, and performs immediate and machine review when the doctor advice or prescription is issued and saved. When the medication warning information rule is not violated or only the risk-free warning level rule is generated, the doctor advice or prescription will automatically pass the review of the medication decision support system. When the high-risk warning level rule is violated, the system will feed back the corresponding warning information to the doctor advice check end, and the checker will make a corresponding manual evaluation according to the warning content to assess whether the doctor advice or prescription needs to be called back. If it needs to be called back, the doctor advice or prescription issuer can receive the warning information prompt in the computer operation end, and can choose to modify or force it through according to the warning information of the called back doctor advice or prescription; when the level warning rule is violated, the system will automatically call back the prescription/doctor advice, and realize the mandatory automatic interception function.

Risk-free warning level rules, high-risk warning level rules and forbidden level warning rules are set according to the safety of medication. Specifically, risk-free warning level rules are warning rules that will not have adverse effects on patients' subjective and objective characteristics according to instructions, clinical medication guidelines or clinical experience; high-risk warning rules are warning rules that will have adverse effects on patients' subjective and objective characteristics according to the instructions, clinical medication guidelines or clinical experience, but there are no prohibitive rules, such as those that belong to the precautions specified in the instructions, but are not taboo; the forbidden level warning rules are warning rules that will have serious adverse effects on the subjective and objective characteristics of patients according to the instructions, clinical medication guide or clinical experience, such as the incompatibility content specified in the instructions. In addition, the serious adverse effects here can be the situation of disability or death.

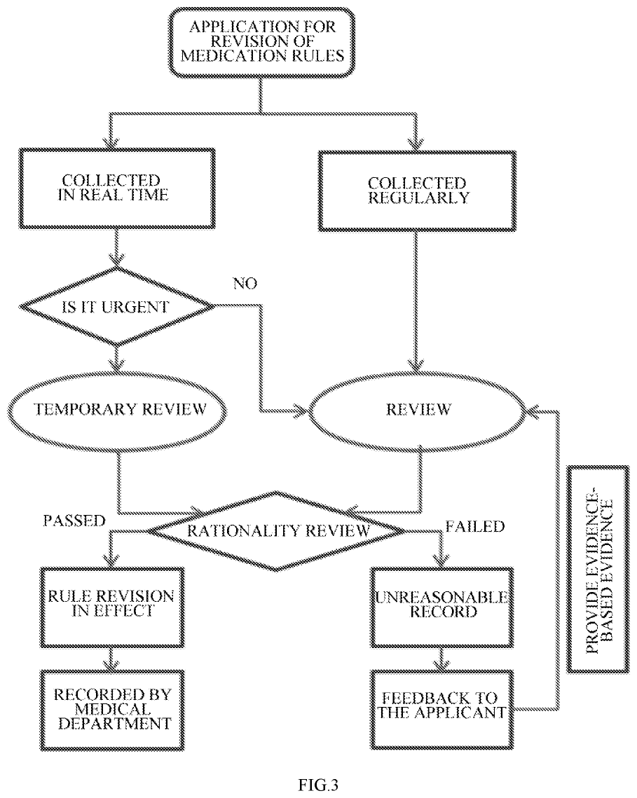

The medication warning information rules are defined by medication warning information entities, including initial rules and revised rules. The initial rules are obtained through drug instructions and medication guides, and the revised rules can be set through the operation process as shown in FIG. 3. Specifically, doctors, nurses and pharmacists submit the application for revision of medication rules, and the doctor advice reviewers collect the application for revision of medication rules in real time and regularly. For the application for revision of medication rules collected in real time, the reviewer judges whether it is an urgent application. If it is an urgent application, the team to which the reviewer belongs will immediately organize the on-the-job staff to review the rationality of the medication rule revision application. If it is judged to be reasonable, the medication rule revision information will be maintained in the graphics state machine and reported to the medical administrative department of the institution where it is located for the record. The whole process needs to be completed within 2 hours, so as to meet the requirements of clinical emergency. If it is not an urgent application, the rationality of the rules will be discussed and reviewed by the team to which the doctor advice reviewers belong together with the regularly collected application for modification of medication rules. In addition, the doctor advice reviewer shall record the application for revision of medication rules that failed the rationality review of the rules, and feed it back to the applicant. If the applicant does not approve of the review results, it can submit evidence-based medicine evidence and re-submit it for review. The doctor advice reviewer shall be an experienced pharmacist, and the experienced pharmacist refers to the reviewing pharmacist or clinical pharmacist with more than 5 years' working experience. In addition, for emergency application, it is necessary to re-examine the routine rationality after clinical emergency use to meet the rationality of medication rules.

The present disclosure also provides an information system of a man-machine integrated doctor advice review mode based on the medication decision support method based on graphics state machine. The information system is divided into two modules, both of which adopt a B/S architecture and can be browsed by a browser, so it is not necessary to deploy at the doctor's workstation. One of the modules is a doctor advice input platform, written in JAVA language, in which doctor advices or prescriptions can be directly input into the system, including: patient medication information, patient visit related information, course records, medication consultation statements, and access to basic business information of doctors, patient inspection information, image data, pathological data, etc.; the second is a medication decision support method system, which is designed by linux operating system and embedded knowledge graph system. It is composed of rule entities and exchanges data with the doctor advice input platform through an interface. The knowledge graph is used to store the edited visual graphic rule entities; the medication decision support method system analyzes the graphic rules in the embedded knowledge graph system through logical data rules, and then judges the accuracy of the doctor advice information, and returns a judgment result to the doctor advice input platform. The doctor advice issuing staff and doctor advice reviewer can both use different computer terminals to call the rational medication rules of embedded knowledge graph, and the medication decision support method system will automatically make judgments, and at the same time, the judgment results will be fed back to the computer terminals to realize remote prescription/doctor advice review and medication decision assistance.

The man-machine integrated doctor advice review mode based on the graphics state machine medication decision support method classifies and clinically verifies the rational medication rules by integrating various scattered medication rule data and clinical medication experience, and associates with the information of disease severity, onset course, doctor's professional level and the like related to patient medication to construct three medication decision indicating graphics state machines, forming a graphics state machine set that accurately and quickly responds to target events, The simple judgment of complex medication logic and the rapid and accurate push of medication decision information can be realized, thus changing the frequent and extensive push mode of medication decision information in the past, and improving the accurate auxiliary decision-making ability of the medication decision support system.

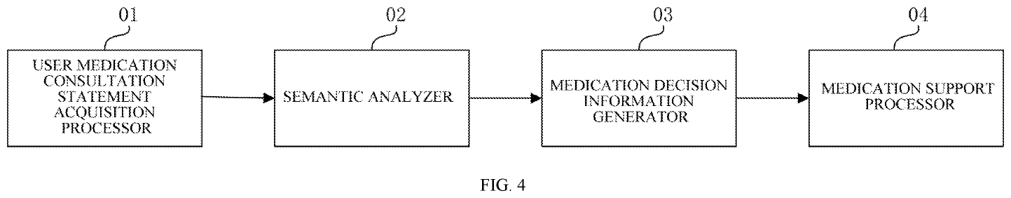

The second aspect of the present disclosure provides a medication decision support apparatus based on a graphics state machine, as shown in FIG. 4, including:

A user medication consultation statement acquisition processor 01 configured to acquire a user medication consultation statement.

A semantic analyzer 02 configured to extract a symptom information entity, an allergy information entity and an disease onset information entity from the medication consultation statement through word segmentation and semantic recognition;

A medication decision information generator 03 configured to generate medication decision information by using the symptom information entity, the allergy information entity and the disease onset information entity based on a preset medication decision indicating graphics state machine set, wherein the medication decision indicating graphics state machine set includes at least one medication decision indicating graphics state machine;

A medication support processor 04 configured to provide primary available drugs for the user according to the medication decision information.

The allergy information entity includes an allergic drug entity and an allergic food entity.

The disease onset information entity includes an age level entity, a weight entity, a height entity, an onset time entity and a disease severity entity.

The medication decision information includes drug name information, drug dose information and medication frequency information.

Specifically, the apparatus obtains the user's medication consultation statement through voice input or graphic input, converts the voice or picture into the text information of the user's medication consultation statement, segments the text information of the medication consultation statement, and segments all possible words that match the lexicon, then uses a statistical language model to determine an optimal segmentation result, and then carries out part-of-speech tagging to generate a symptom information entity, an allergy information entity and an disease onset information entity. This information is input into the medication decision information generator, and medication decision information is generated according to the medication decision indicating graphics state machine set. The medication support processor can regularly remind users of medication according to medication frequency, including drug name and drug dose.

On the other hand, the apparatus may also obtain a medication consultation statement of a user through voice input or graphic input, and then converts the medication consultation statement into entity participles by a semantic analyzer, and corrects them by an emotional score and neural network model; takes this as input, uses the first medication decision indicating graphics state machine to determine the drug names of various available drugs, which are input to the second medication decision indicating graphics state machine, optimizes the drugs to be administered according to the patient's allergy entity information; and then further inputs them to the third medication decision indicating graphics state machine to obtain medication decision information; wherein the decision information includes drug name, drug dose and medication frequency.

On the other hand, the apparatus obtains the objective information such as the user's doctor advice, inspection, examination, pathology, image, etc. through voice input or graphic input, thus forming the subjective information of the medication consultation statement of the user and the objective information of the doctor advice, inspection, examination, etc., which are converted into entity participles by the semantic analyzer; based on preset medication decision indicating graphics state machine set, the first medication decision indicating graphics state machine is used to determine the drug names of various available drugs and the doctor advice information with risk warning, which are input to the second medication decision indicating graphics state machine, and the drug varieties are optimized according to the patient's allergy entity information, and then input to the third medication decision indicating graphics state machine to obtain medication decision information; the decision information includes the recommended drug name, drug dose and medication frequency, and drug warning information ordered by doctors.

On the other hand, the apparatus obtains basic information and professional level information of doctors, as well as objective information such as user's doctor advice, inspection, examination, pathology, image, etc. through voice input or graphic input, so as to form subjective information of medication consultation statement of the user, objective information of doctor advice, inspection, examination, etc., and characteristic information of doctor's professional level, which are converted into entity participles by the semantic analyzer; based on preset medication decision indicating graphics state machine set, the first medication decision indicating graphics state machine is used to determine the drug names of various available drugs and the doctor advice information with risk warning, which are input to the second medication decision indicating graphics state machine. According to the patient's allergy entity information, doctor's professional level entity information and conflict package entity information, and further through the setting of doctor's professional level entity and allowing dynamic update of medication warning information according to the conflict package entity recorded by doctor's operation, the recommended medication varieties and doctor advice warning information are optimized and input to the third medication decision indicating graphics state machine, so as to obtain medication decision information. The decision information includes the drug name, drug dose and frequency of recommended drugs, and medication warning information related to the doctor's decision level.

The medication decision support apparatus based on a graphics state machine comprehensively considers the subjective information of the medication consultation statement of the user and the objective information of doctor advice, inspection, examination, pathology and image in the setting process of three medication decision indicating graphics state machines, and jointly forms the support for medication decision. Furthermore, through the setting of the doctors' professional level entity and the setting of the conflict package entity that allow dynamic updating according to doctors' operation records, only the medication decision support information related to doctors' decision level is recommended, thus avoiding the alarm fatigue of the clinical decision support system to doctors, and thus meeting the requirements of accurate drug decision support and graded recommendation

In addition, the three graphics state machines interact with each other and are arranged in series through a plurality of common entities, so that the medication recommendation results can be obtained according to entities with multiple dimensions or attributes by combining the medication rule grades and individual deviation correction. Moreover, the medication recommendation results among different entities can be interrelated, verified and graded by a plurality of common entities, and complement each other, so as to achieve more accurate and quick response clinical medication decision support from different dimensions, and further meet the more stringent requirements of clinical diagnosis and treatment medication decision support.

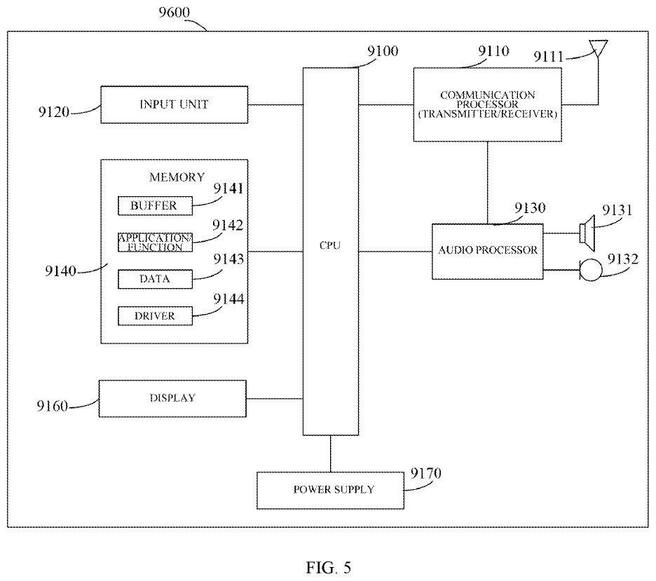

Please refer to FIG. 5, which is a schematic block diagram of the system configuration of the medication decision support device 9600 (hereinafter referred to as the electronic device 9600) based on a graphics state machine according to an embodiment of the present disclosure. As shown in FIG. 5, the electronic device 9600 may include a CPU 9100 and a memory 9140; the memory 9140 is coupled to the CPU 9100. Note that this FIG. 5 is exemplary. Other types of structures can also be used to supplement or replace this structure to realize telecommunication functions or other functions.

In another embodiment, the medication decision function can be integrated into the CPU 9100. For example, the CPU 9100 can be configured to perform the following control:

S01: obtaining a medication consultation statement of a user;

S02: extracting the symptom information entity, the allergy information entity and the disease onset information entity from the medication consultation statement through word segmentation and semantic recognition;

S03: generating medication decision information by using the symptom information entity, the allergy information entity and the disease onset information entity based on a preset medication decision indicating graphics state machine set, wherein the medication decision indicating graphics state machine set includes at least one medication decision indicating graphics state machine;

S04: providing primary available drugs for the user according to the medication decision information.

In another embodiment, the medication decision function can be integrated into the CPU 9100. For example, the CPU 9100 can be configured to perform the following control:

S01: obtaining a medication consultation statement of the user, basic information of doctors and the user's doctor advice, inspection, examination, pathology and image information;

S02: extracting the symptom information entity, allergy information entity, disease onset information entity, medication warning information entity, doctor advice information entity, inspection information entity, examination information entity, pathological information entity, image information entity, doctor's professional level entity and conflict package entity from the medication consultation statement, doctor's basic information and doctor advice, examination, examination, pathology and image information through word segmentation and semantic recognition;

S03: generating medication decision information based on the preset medication decision indicating graphics state machine set by using the symptom information entity, allergy information entity, disease onset information entity, medication warning information entity, doctor advice information entity, inspection information entity, examination information entity, pathological information entity, image information entity, doctor's professional level entity and conflict package entity; the medication decision indicating graphics state machine set includes at least one medication decision indicating graphics state machine;

S04: providing primary available drugs and warning information of the doctor advice for the user according to the medication decision information.

As can be seen from the above description, the electronic device provided by the embodiment of the present disclosure generates medication decision information by using the acquired symptom information entity, allergy information entity and disease onset information entity based on the preset medication decision indicating graphics state machine set, and reminds the user to take medication according to the specification according to the medication time, so as to avoid wrong or missed medication, which can better support the user's medication, ensure the user's medication safety, and improve the therapeutic effect of drugs.

In another embodiment, the medication decision support apparatus can be configured separately from the central processor 9100. For example, the medication decision support apparatus can be a chip connected to the central processor 9100, and the medication decision can be realized by the control of the central processor.

As shown in FIG. 3, the electronic device 9600 may further include a communication processor 9110, an input unit 9120, an audio processor 9130, a display 9160, and a power supply 9170. Note that the electronic device 9600 does not necessarily include all the components shown in FIG. 3. In addition, the electronic device 9600 may also include components not shown in FIG. 3, which can refer to the prior art.

As shown in FIG. 3, the CPU 9100, sometimes referred to as a controller or operation control, may include a microprocessor or other processor devices and/or logic devices, and the CPU 9100 receives inputs and controls the operations of various components of the electronic device 9600.

The memory 9140 can be, for example, one or more of a buffer, a flash memory, a hard drive, a removable medium, a volatile memory, a nonvolatile memory or other suitable devices. The above information related to failure can be stored, and in addition, the program for executing the information can be stored. And the CPU 9100 can execute the program stored in the memory 9140 to realize information storage or processing.

The input unit 9120 provides input to the CPU 9100. The input unit 9120 is, for example, a key or a touch input device. The power supply 9170 is used to supply power to the electronic device 9600. The display 9160 is used to display objects such as images and characters. The display can be, but not limited to, for example, an LCD display.

The memory 9140 may be a solid-state memory, such as a read only memory (ROM), a random access memory (RAM), a SIM card, etc. It may also be a memory that holds information even when power is turned off, can be selectively erased, and is provided with more data. Examples of this memory are sometimes called EPROM, etc. The memory 9140 can also be some other type of apparatus. The memory 9140 includes a buffer memory 9141 (sometimes called a buffer). The memory 9140 may include an application/function storage part 9142 for storing application programs and function programs or a flow for executing the operation of the electronic device 9600 through the CPU 9100.

The memory 9140 may also include a data storage part 9143 for storing data, such as user information, digital data, pictures, sounds and/or any other data used by electronic devices. The driver storage part 9144 of the memory 9140 may include various drivers for the communication function of the electronic device and/or for executing other functions of the electronic device, such as messaging application, address book application, etc.

The communication processor 9110 is a transmitter/receiver 9110 that transmits and receives signals via an antenna 9111. The communication processor (transmitter/receiver) 9110 is coupled to the CPU 9100 to provide input signals and receive output signals, which may be the same as the case of a conventional mobile communication terminal.

Based on different communication technologies, multiple communication processors 9110, such as a cellular network module, a Bluetooth module and/or a wireless LAN module, can be provided in the same electronic device. The communication processor (transmitter/receiver) 9110 is also coupled to a speaker 9131 and a microphone 9132 via an audio processor 9130 to provide audio output via the speaker 9131 and receive audio input from the microphone 9132, thereby realizing a general telecommunications function. The audio processor 9130 may include any suitable buffer, decoder, amplifier, etc. In addition, the audio processor 9130 is also coupled to the central CPU 9100, so that the audio can be recorded on the local machine through the microphone 9132, and the sound stored on the local machine can be played through the speaker 9131.

An embodiment of the present disclosure also provides a computer-readable storage medium which can realize all the steps in the medication decision support method in the above embodiment, and the computer-readable storage medium stores a computer program which, when executed by a processor, can realize all the steps in the medication decision support method in the above embodiment, in which the executing agent is a server or a client.

As can be seen from the above description, the computer-readable storage medium provided by the embodiment of the present disclosure generates medication decision information by using the acquired symptom information entity, allergy information entity and disease onset information entity based on the preset medication decision indicating graphics state machine set, wherein, through the association of three independent first medication decision indicating graphics state machines, second medication decision indicating graphics state machines and third medication decision indicating graphics state machines, a graphics state machine set with a fast response speed to target events is formed; in addition, the graphics state machine set constructed fully considers the influence of disease information on drug dose and medication frequency through an emotional score, so as to improve the accuracy of drug recommendation. Finally emotional discrimination and customized neural network model are used to analyze the disease degree of the user's input statement, and then compare it with the disease degree of the user's statement, the corrected value of the disease degree is obtained. By using the corrected value, the excessive judgment or underestimation of the disease degree caused by the user's emotional factors can be eliminated, which can better support the user's medication, ensure the user's medication safety and improve the therapeutic effect of the drug.

It should be understood by those skilled in the art that embodiments of the present disclosure can be provided as methods, apparatuses, or computer program products. Therefore, the present disclosure may take the form of an entirely hardware embodiment, an entirely software embodiment, or an embodiment combining software and hardware aspects. Furthermore, the present disclosure may take the form of a computer program product implemented on one or more computer usable storage media (including but not limited to magnetic disk storage, CD-ROM, optical storage, etc.) containing computer usable program code therein.

The present disclosure is described with reference to flowcharts and/or block diagrams of methods, devices (apparatuses), and computer program products according to embodiments of the present disclosure. It should be understood that each flow and/or block in flowchart and/or block diagram, and combinations of flows and/or blocks in flowchart and/or block diagram can be realized by computer program instructions. These computer program instructions may be provided to the processor of a general-purpose computer, a special-purpose computer, an embedded processor or other programmable data processing equipment to produce a machine, so that the instructions executed by the processor of the computer or other programmable data processing equipment generate an apparatus for implementing the functions specified in one or more flow charts and/or one or more blocks of the block diagram.

These computer program instructions can also be stored in a computer-readable memory that can direct a computer or other programmable data processing equipment to work in a specific way, so that the instructions stored in the computer-readable memory produce an article of manufacture including an instruction apparatus that implement the functions specified in one or more flow charts and/or one or more blocks of the block diagrams.

These computer program instructions can also be loaded on a computer or other programmable data processing equipment, so that a series of operation steps are executed on the computer or other programmable equipment to produce a computer-implemented process, so that the instructions executed on the computer or other programmable equipment provide steps for realizing the functions specified in one or more flows of the flowchart and/or one or more blocks of the block diagram.

The steps of the method or algorithm described combined with the embodiments of the present disclosure may be implemented in a hardware manner, or may be implemented in a manner in which a processor executes software instructions. The software instructions may consist of corresponding software modules, and the software modules can be stored in Random Access Memory (RAM), flash memory, Read Only Memory (ROM), Erasable Programmable ROM (EPROM), Electrically Erasable Programmable Read-Only Memory (EEPROM), registers, hard disks, removable hard disks, CD-ROMs or any other forms of storage media well-known in the art. An exemplary storage medium is coupled to the processor, such that the processor can read information from, and write information to, the storage medium. The storage medium can also be an integral part of the processor. The processor and storage medium may reside in an Application Specific Integrated Circuit (ASIC). Alternatively, the ASIC may be located in a node device, such as the processing node described above. In addition, the processor and storage medium may also exist in the node device as discrete components.

It should be noted that when the data compression apparatus provided in the foregoing embodiment performs data compression, division into the foregoing functional modules is used only as an example for description. In an actual application, the foregoing functions can be allocated to and implemented by different functional modules based on a requirement, that is, an inner structure of the apparatus is divided into different functional modules, to implement all or some of the functions described above. For details about a specific implementation process, refer to the method embodiment. Details are not described herein again.

All or some of the foregoing embodiments may be implemented by using software, hardware, firmware, or any combination thereof. When the software is used for implementation, all or some of the embodiments may be implemented in a form of a computer program product. The computer program product includes one or more computer instructions. When the computer program instructions are loaded and executed on a server or a terminal, all or some of the procedures or functions according to the embodiments of this application are generated. The computer instructions may be stored in a computer-readable storage medium or may be transmitted from a computer-readable storage medium to another computer-readable storage medium. For example, the computer instructions may be transmitted from a web site, computer, server, or data center to another web site, computer, server, or data center in a wired (for example, a coaxial optical cable, an optical fiber, or a digital subscriber line) or wireless (for example, infrared, radio, or microwave) manner. The computer-readable storage medium may be any usable medium accessible by a server or a terminal, or a data storage device, such as a server or a data center, integrating one or more usable media. The usable medium may be a magnetic medium (for example, a floppy disk, a hard disk, or a magnetic tape), an optical medium (for example, a digital video disk (DVD)), or a semiconductor medium (for example, a solid-state drive).

In the present disclosure, specific examples are used to explain the principle and implementation of the present disclosure, and the explanations of the above examples are only used to help understand the method and core ideas of the present disclosure; At the same time, according to the idea of the present disclosure, there will be changes in the specific implementation and application scope for those skilled in the field. To sum up, the contents of this specification should not be construed as a limitation of the present disclosure.

What is claimed is:

1. A medication decision support method based on a graphics state machine, characterized by comprising:
acquiring a medication consultation statement of a user;
extracting a symptom information entity, an allergy information entity and an disease onset information entity from the medication consultation statement through word segmentation and semantic recognition;
generating medication decision information by using the symptom information entity, the allergy information entity and the disease onset information entity based on a preset medication decision indicating graphics state machine set, wherein the medication decision indicating graphics state machine set comprises at least one medication decision indicating graphics state machine;
providing primary available drugs for the user according to the medication decision information;
wherein the allergy information entity comprises an allergic drug entity and an allergic food entity;
the disease onset information entity comprises an age level entity, a weight entity, a height entity, an onset time entity and a disease severity entity;
the medication decision information comprises drug name information, drug dose information and medication frequency information.

2. The medication decision support method according to claim 1,
wherein the medication decision indicating graphics state machine set comprises a first medication decision indicating graphics state machine, a second medication decision indicating graphics state machine and a third medication decision indicating graphics state machine;
the first medication decision indicating graphics state machine comprises the symptom information entity, a diagnosis entity, a drug indication entity, and a symptom-diagnosis-drug indication correlation;
the second medication decision indicating graphics state machine comprises the allergic drug entity, the allergic food entity, a correlation of cross allergy between drugs, and a correlation of cross allergy between drugs and food;
the third medication decision indicating graphics state machine comprises the age level entity, the weight entity, the height entity, the onset time entity, the disease severity entity, and a correlation between an age level, a weight, a height, an onset time, a disease degree and a dosage and a medication frequency;
the first, second and third medication decision indicating graphics state machines have a plurality of common entities; wherein the medication decision graphics state machine comprises a definition of different clinical event entities, a definition of multiple attributes of the entities and a correlation between the entities; the graphics state machine is preset with general medical logic processors, and each general medical logic processor contains an applicable logic; the relationship between the entities in the medication decision graphics state machine can be inferred based on the applicable logic, and a medication recommendation result can be obtained according to individual inputs defined by the multiple attributes of the entities.

3. The medication decision support method according to claim 1, wherein the disease onset information entity comprises disease severity information, and the method further comprises:
analyzing medication consultation statement of the user based on the emotion word segmentation dictionary, and generating an emotional score of the consultation statement, wherein the emotional score is divided into different emotional tendency grades, which is specifically as follows:
dividing the medication consultation statement of the user into an emotional verb W_{V} and an emotional adverb W_{adj} based on the emotion word segmentation dictionary; matching a current emotional verb W_{V} with an emotion dictionary, if the current emotional verb W_{V} is a positive word, an emotional value is 1, if the current emotional verb W_{V} is a negative word, the emotional value is −1; and matching an emotional adverb W_{adj} with the emotion dictionary, and if the emotional adverb W_{adj} is a positive word, the emotional value is 1, if the emotional adverb W_{adj} is a negative word, the emotional value is −1;
calculating a cumulative tendency score of each emotional verb W_{V}:
α=Σᵢ₌₁ⁿWvi (1)
where α>0 indicates that an action emotional tendency is of a positive feedback type, which is recorded as positive −α, α<0 indicates that the action emotional tendency is of a negative feedback type, which is recorded as negative −α, α=0 indicates that the action emotional tendency is of a neutral type, which is recorded as neutral −α,
calculating a cumulative tendency score of each emotional adverb W_{adj} is calculated:
β=Σᵢ₌₁ⁿWadji (2)
where β>0 indicates that an action emotional tendency is of a positive feedback type, which is recorded as positive-β; β<0 indicates that the action emotional tendency is of a negative feedback type, which is recorded as negative-β, β=0 indicates that the action emotional tendency is of a neutral type, which is recorded as neutral-β,
generating a specific emotional tendency grade and a corresponding emotional score according to the expressions of the emotional verb W_{V} and the emotional adverb W_{adj}; and
correcting the disease severity information by using the emotional score.

4. The medication decision support method according to claim 3, wherein the step of correcting the disease severity information by using the emotional score comprises:
generating estimated disease severity information according to the emotional score, which comprises the following steps of: calculating the estimated disease severity information by using historical diagnosis data of hospital electronic cases in recent N years, wherein the estimated disease severity information comprises mild diseases, moderate diseases, severe diseases and critical diseases, and each estimated disease severity information is obtained by a decision tree generated by the electronic case data of related diagnosis, and further decision tree nodes are distinguished by emotional tendency grades;
comparing the estimated disease severity information with the disease severity information in the medication consultation statement of the user to generate an emotion corrected value; and
correcting the disease severity information according to the emotion corrected value.

5. The medication decision support method according to claim 4, wherein the step of comparing the estimated disease severity information with the disease severity information in the medication consultation statement of the user to generate an emotion corrected value comprises:
comparing the estimated disease severity information with the disease severity information in the medication consultation statement of the user to generate a difference score; and
generating the emotion corrected value based on the difference score and a disease degree of the disease severity information in the medication consultation statement of the user.

6. The medication decision support method according to claim 4, wherein the step of correcting the disease severity information according to the emotion corrected value comprises:
inputting the emotion corrected value and the disease severity information in the medication consultation statement of the user into a neural network model corresponding to the target user, wherein an output of the neural network model is the estimated disease severity information;
wherein the neural network model is trained by using historical consultation statements of patients with the same diagnosis as the user and actual disease severity information; the disease severity information comprises mild, moderate, severe and critical degrees; the patients with the same diagnosis comprise patients with a same diagnosis, a same age level and a same education level.

7. The medication decision support method according to claim 6, wherein the step of training the neural network model by using historical consultation statements of patients with the same diagnosis as the user and actual disease severity information comprises:
de-noising the historical consultation statements of the patients with the same diagnosis, the age level and education level as the target user;
performing emotion analysis on the denoised historical consultation statements based on the emotion dictionary to obtain corresponding emotional scores; wherein the emotion dictionary is obtained by expanding a Hownet emotion dictionary and simplified Chinese NTUSD dictionary by combining a basic emotion dictionary, and a method of expanding the emotion dictionary is mainly based on semantic similarity and synonym methods;
marking the corresponding historical consultation statements with the emotional scores, and forming training data by combining the actual disease severity information; and
training the neural network model by using a training set including a plurality of the training data.

8. A medication decision support apparatus based on a graphics state machine, comprising:
a user medication consultation statement acquisition processor configured to acquire a user medication consultation statement;
a semantic analyzer configured to extract a symptom information entity, an allergy information entity and an disease onset information entity from the medication consultation statement through word segmentation and semantic recognition;
a medication decision information generator configured to generate medication decision information by using the symptom information entity, the allergy information entity and the disease onset information entity based on a preset medication decision indicating graphics state machine set, wherein the medication decision indicating graphics state machine set comprises at least one medication decision indicating graphics state machine;
a medication support processor configured to provide primary available drugs for the user according to the medication decision information;
wherein the allergy information entity comprises an allergic drug entity and an allergic food entity;
the disease onset information entity comprises an age level entity, a weight entity, a height entity, an onset time entity and a disease severity entity; and
the medication decision information comprises drug name information, drug dose information and medication frequency information.

9. A medication decision support device based on a graphics state machine, comprising a memory, a processor, a communication processor, and a computer program stored in the memory and operable on the processor, wherein the processor implements the medication decision support method based on a graphics state machine according to claim 1 when executing the program.

10. A computer-readable storage medium on which a computer program is stored, wherein the computer program, when executed by a processor, realizes the medication decision support method based on a graphics state machine according to claim 1.
